(19) United States
(12) Patent Application Publication
(10) Pub. No.: US 2009/0036496 A1
(43) Pub. Date: Feb. 5, 2009

(54) Treatment of Stress Urinary Incontinence and Mixed Urinary Incontinence

(75) Inventors: Hugues Bienayme, St. Symphorien D'Ozon (FR); Jacques Ferte, Issy-Les-Moulineaux (FR)

Correspondence Address: OLIFF & BERRIDGE, PLC, P.O. BOX 320850, ALEXANDRIA, VA, 22320-4850, US

(73) Assignee: UROGENE, Paris (FR)

(21) Appl. No.: 12/083,233

(22) PCT Filed: Aug. 28, 2006
(86) PCT No.: PCT/IB2006/003682

§ 371 Date: Apr. 25, 2008

Related U.S. Application Data
(60) Provisional application No. 60/727,839, filed on Oct. 19, 2005.

(30) Foreign Application Priority Data
Oct. 19, 2005 (FR) 05/10649

(51) Int. Cl. A61K 31/4439; A61P 13/10

(52) U.S. Cl. 514/339

(57) ABSTRACT

Use of a compound corresponding to formula (I): in which R represents hydrogen or a group chosen from alkyl, alkylene, alkylidyne, cycloalkyl, cycloalkylene, cycloalkylidyne and —CONH₂ groups, and —COR′ and —COOR′ groups, where R′ is chosen from alkyl, alkylene, alkylidyne, cycloalkyl, cycloalkylene and cycloalkylidyne groups, it being possible for said groups R and/or R′ to be substituted and/or interrupted with —O—, —COO—, —OCO—, —NHCO— or —CONH— functions, or a pharmaceutically acceptable salt of said compound, for obtaining a medicinal product for use in the treatment of stress urinary incontinence and of mixed urinary incontinence.

The present invention relates to the urological applications of N-(4-pyridinyl)-1H-indol-1-amine compounds, and more particularly to the use thereof in the treatment of stress urinary incontinence or of mixed incontinence.

The function of the lower urinary tract is to store and, when appropriate, release urine. Briefly, the bladder is a smooth muscle reservoir (the detrusor) that passively distends with filling. Closure of the bladder during the filling phase is secured by contraction of the urethral smooth muscle and of the external striated sphincter (rhabdosphincter). The lower urinary tract functions through a system of highly coordinated processes that involve the control of smooth and skeletal muscles of the bladder and urethra, by both central and peripheral nervous systems [Burgard et al, “New pharmacological treatments for urinary incontinence and overactive bladder”, Curr. Opin. Investig. Drugs. 6, 81-89 (2005)].

Under normal conditions, sensory information regarding bladder filling is primarily transmitted to the central nervous system (CNS) via Aδ sensory afferent fibers. When bladder volume has reached a critical threshold, and micturition is behaviourally and environmentally appropriate, a spinobulbospinal reflex is activated which results in the release of acetylcholine at the bladder neuromuscular junction, producing a bladder contraction. Simultaneously, the urethra opens, following relaxation of both the smooth muscle and the rhabdosphincter and allows expulsion of the stored urine.

Impairment in the ability to store urine results in conditions which can be separated into two main disorders with completely distinct underlying dysfunctions: stress urinary incontinence and urge-related disorders.

Stress incontinence is a loss of urine in response to a cough, laugh, sneeze or any other physical activity that increases intra-abdominal pressure. The underlying pathology of stress urinary incontinence often involves the rhabdosphincter.

Overactive bladder (OAB) and the related condition of urge urinary incontinence represent the other major disorder of the storage function. They are characterized by a frequent need to void (frequency), with an intense urge to do so (urgency) and a need to void during the night (nocturia). Contrary to stress urinary incontinence, OAB and urge incontinence are not associated with urethral sphincter control but rather involves disturbances in bladder functions and the regulation thereof.

Mixed urinary incontinence incorporates symptoms of both stress and urge.

Currently, the pharmacological treatments for stress and urge urinary incontinence are completely distinct. Several options exist for urge incontinence/OAB, the mainstay being antimuscarinics.

The therapeutic approaches for stress urinary incontinence are based on perineal-sphincter rehabilitation, alone or combined with biofeedback or with electrostimulation. In the case of very debilitating stress urinary incontinence a surgical treatment can be proposed (Prise en charge de l'incontinence urinaire de la femme en médecine générale [Management of female urinary incontinence in general medicine], Recommendations of the ANAES, May 2003).

Until recently, there was no frontline pharmacological treatment for stress incontinence. If rehabilitation fails, oestrogen replacement therapy and/or the use of alpha-adrenergic compounds can be prescribed in menopausal women.

The objective of the pharmacological approaches for the treatment of stress incontinence is to increase urethral tone.

The only treatment approved specifically for stress incontinence is duloxetine (Yentreve), a mixed inhibitor of serotonin uptake and norepinephrine uptake. Preclinical and clinical studies have shown that this compound enhances urethral closure and conserves bladder-sphincter coordination. On the other hand, this compound has adverse effects which greatly limit the use thereof.

Urinary incontinence has a harmful impact on the daily life of the individuals who suffer therefrom, and medicinal products that are more effective and have a better benefit/risk ratio are still being sought.

The subject of the present invention lies in the use of a family of compounds for obtaining a medicinal product for use in the treatment of stress urinary incontinence and of mixed incontinence.

The compounds of the invention correspond to formula (I):

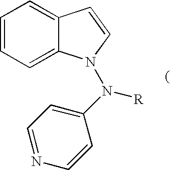

in which R is chosen from alkyl, alkylene, alkylidyne; cycloalkyl, cycloalkylene, cycloalkylidyne and —CONH₂ groups, and —COR′ and —COOR′ groups, where R′ is chosen from alkyl, alkylene, alkylidyne, cycloalkyl, cycloalkylene and cycloalkylidyne groups, it being possible for said groups R and/or R′ to be substituted and/or interrupted with —O—, —COO—, —OCO—, —NHCO— or —CONH— functions.

The pharmaceutically acceptable salts of these compounds are also part of the invention. It may in fact be preferable to prepare, purify and/or store a salt corresponding to the active compound, for example a pharmaceutically acceptable salt. Examples of pharmaceutically acceptable salts are given in the publication Berge et al., “Pharmaceutically acceptable salts”, J. Pharm. Sci., 66, 1-19 (1977). By way of examples, mention may be made of salicylic, hydrochloric and fumaric salts.

According to an advantageous variant, the invention relates to the compounds of N-(4-pyridinyl)-1H-indol-1-amine type of general structure (I) when the group R is equal to a hydrogen atom, N-(4-pyridinyl)-1H-indol-1-amine (compound HP748), or to the n-propyl group, N-propyl-N-(4-pyridinyl)-1H-indol-1-amine or besipirdine (compound HP749), and also the pharmaceutically acceptable salts thereof, and more particularly the use thereof for obtaining a medicinal product for use in the treatment of symptoms associated with stress incontinence and mixed incontinence.

According to the invention, two compounds mentioned above can advantageously be associated or combined. A preferred association or combination comprises a compound of formula (I) in which R represents a hydrogen atom (HP748) and a compound of formula (I) in which R represents the n-propyl group (HP749), in the knowledge that the compound HP748 can be obtained by metabolization of the compound HP749 in humans. The combination of actions is particularly favourable for obtaining a medicinal product for use in the treatment of symptoms associated with stress incontinence and mixed incontinence.

The compounds (I) of the invention can be obtained by means of a process such as that described in U.S. Pat. No. 4,970,218.

The examples hereinafter illustrate the effect of the compounds according to the invention and the advantage of the latter compared with known compounds that find applications in the urological field. Thus, the compounds tested are those described in the table below, where the compound HP184 is that described in document WO 02/064126, and the compound HP183 is a metabolite of HP184.

TABLE

| Compound tested | Form | Formula |
|---|---|---|
| HP748 (compound I of theinvention where R representsH) | Salicylate | 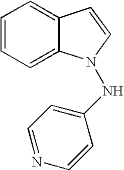 |
| HP749 (compound I of theinvention where R representsCH₂—CH₂—CH₃) | Hydrochloride | 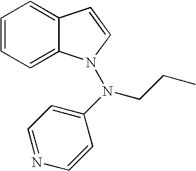 |
| HP183 (metabolite ofHP184) | Hydrochloride | 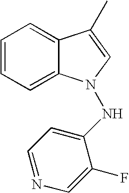 |
| HP184 | Fumarate | 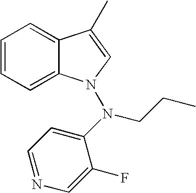 |

The compounds were tested in ex vivo experiments carried out on rabbit urethra.

### EXAMPLE 1

### Effect of the Compounds on Neurogenic Contractions of the Urethra

The tests were carried out on isolates of rabbit urethra which were obtained as follows.

The whole urethra was excised and washed in order to remove the adipose and connective tissues, and then cut into rings approximately 4 mm in diameter, one at the median level, the other at the distal level of the urethra.

1.1 Effect on Contractions by Activation of Alpha1-Adrenergic Receptors

In this trial, the ability of each of the four compounds mentioned above to induce, by themselves, a contraction of the urethra by activation of the alpha1-adrenergic receptors, compared with norepinephrine used as a positive control, is measured.

Description of the Trial Protocol:

The protocol followed is an adaptation of the protocol described by Van der Graaf et al. (Eur. J. Pharmacol., 327: 25-32, 1997).

Krebs-Henseleit solution was modified by adding propanolol (1 μM), normetanephrin (1 μM), desipramine (0.1 μM) and deoxycorticosterone (3 μM) in order to block the beta-adrenergic receptors, catechol-O-methyltransferase and the uptake of noradrenalin type 1 (neuronal) and 2 (extraneuronal), respectively.

An initial force of 1 g is applied to the samples. After a period of 60 minutes of equilibration, the rings are brought into contact with norepinephrine (30 μM), twice consecutively, separated by washing for 60 minutes. The rings that have a contractile response of less than 1 g are discarded and new rings are optionally prepared. After washing for 30 minutes, the compound tested is added at cumulative concentrations ranging from 0.01 to 100 μM. In parallel, the solvent of the compound (distilled water or DMSO) is tested thereon so as to obtain a control curve.

In the case of HP748, an additional experiment was carried out in which prazosin (1 μM), an alpha1-adrenergic receptor antagonist, was incubated for 30 minutes before the addition of HP748.

Results of the Trials

The results are expressed as percentage of the contraction induced by the second addition of norepinephrine at 30 μM as a function of the concentration of the compound. The latter is represented on a logarithmic scale, by the logarithm of the value in mol/l.

They are represented in the following figures:

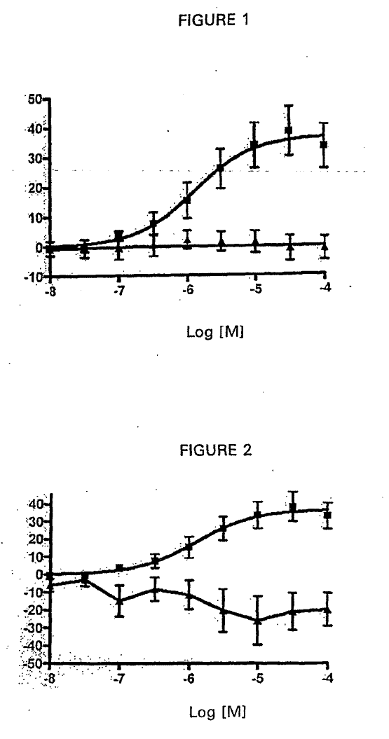

### FIG. 1:

▪ for HP748 (n=5) and ▴ for DMSO (n=5)

### FIG. 2:

▪ for HP748 (n=5) and ▴ for HP748+prazosin (n=4).

The compounds HP749, HP183 and HP184 had no effect.

It is observed that only HP748 induces a significant contraction. This contraction is completely blocked after preincubation with 1 μM of prazosin, confirming that this effect is mediated by alpha1-adrenergic receptors.

1.2 Ability to Potentiate a Contraction Induced by Norepinephrin

In this trial, the ability of the compounds HP749, HP183 and HP184 to potentiate a urethral contraction induced by norepinephrine is measured. It is known that such an activity is characteristic of norepinephrine uptake inhibitors and allows strengthening of urethral tone.

Description of the Trial Protocol:

The protocol followed is an adaptation of the protocol described by Foreman and McNulty (Life Sci. 53: 193-200, 1993).

Krebs-Henseleit solution was modified by adding propanolol (1 μM) and normetanephrin (1 μM) in order to block the beta-adrenergic receptors and catechol-O-methyltransferase, respectively.

An initial force of 1 g is applied to the samples. After a period of 60 minutes of equilibration, the rings are brought into contact with norepinephrine (30 μM), twice consecutively, separated by washing for 60 minutes. The rings that have a contractile response of less than 1 g are discarded and new rings are optionally prepared. After washing for 30 minutes, a dose-response curve is established for each ring, for cumulative concentrations of norepinephrine of 0.01 to 100 μM. After further washing for 30 minutes, each compound tested or its solvent is incubated at a given concentration (1 μM for HP749, and 100 μM for HP183 and HP184) for 30 minutes and a new curve of dose-response to norepinephrine is established.

Results of the Trials:

The results are evaluated by comparing the EC₅₀ values of the norepinephrine dose-response curves before and after the addition of the compound tested or of the control. The protocol was validated by testing tomoxetine, a selective inhibitor of norepinephrine uptake.

The results are represented in the following figures:

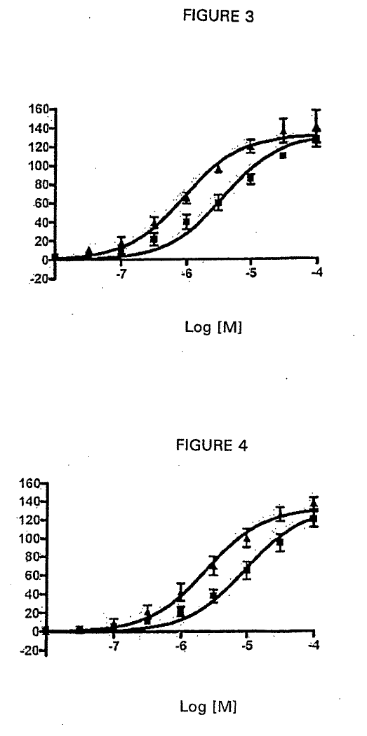

FIG. 3:

▪ for first norepinephrine curve (n=4), and
▴ for norepinephrine+1 μM tomoxetine (n=4)

FIG. 4:

▪ for first norepinephrine curve (n=5), and
▴ for norepinephrine+1 μM HP749 (n=5)

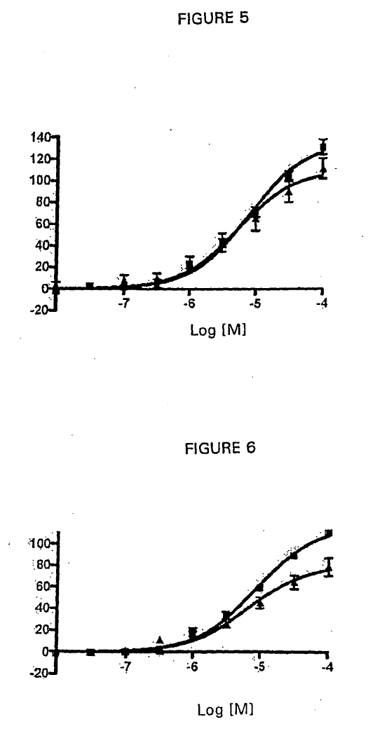

FIG. 5:

▪ for first norepinephrine curve (n=5), and
▴ for norepinephrine+100 μM HP184 (n=5)

### FIG. 6:

▪ for first norepinephrine curve (n=5), and
▴ for norepinephrine+100 μM HP183 (n=5)

It is observed that only HP749 has an effect, significantly decreasing (p<0.0001) the EC₅₀ value of the norepinephrine dose-response curve. In fact, the values of pEC₅₀ (-logEC₅₀) before and after incubation with HP749 are, respectively, 5.02 (4.88-5.15, 95% C.I.) and 5.60 (5.47-5.74, 95% C.I.). Tested in parallel, the solvent of HP749 (distilled water) showed no effect on the EC₅₀ value.

The effect of HP749 is similar to that induced by the reference compound, tomoxetine, the pEC₅₀ value going from 5.43 (5.28-5.57, 95% C.I.) to 6.04 (5.90-6.19; 95% C.I.) before and after the incubation with tomoxetine.

### EXAMPLE 2

### Comparative Effects of HP749 and Duloxetine on Striated Sphincter Function Under Irritated Bladder Conditions

The effects of cumulative doses of HP749 (0, 1, 3 and 5 mg/kg i.v.), or duloxetine (0, 1 and 2 mg/kg i.v.) on the electromyographic and cystometric parameters were evaluated in anaesthetized female New Zealand white rabbits (n=12/group) under irritated conditions (continuous transvesical infusion of 0.5% acetic acid). The study was performed in accordance with the ethical standards of the Helsinki declaration.

Cystometry

Continuous cystometry was performed with the animal supine using a subcutaneous cystostomy. A T tube was connected to the multiperforable plug using a 20-gauge needle, online with a TRA021 pressure transducer and a micro-injection pump. Room-temperature diluted acetic acid was infused into the bladder at a rate of 1.4 ml/hr to elicit repetitive voidings, which allowed collection of data for a large number of voiding cycles. Continuous cystometry was recorded on a PowerLab 4/25.

At the beginning of the cystometry the bladder was emptied. The liquid infusion to stabilize the cyclic voidings was maintained at least for 60 minutes, continuously recording the cystometrogram. Then, placebo was intravenously administered, and the cystometrogram was recorded for other 40 minutes. Consecutive doses of duloxetine or HP749 were then administered with an interval of 40 minutes.

Electromyography of Striated Sphincter (SS-EMG)

Two electrodes (30-gauge needle) were placed percutaneously into the striated anal sphincter approximately 5-10 mm. lateral to the anus. Electrical signals were amplified on an ML136 preamplifier (ADInstruments, PanLab, Barcelona, Spain), filtered below 1 Hz and above 5 kHz and displayed on a PowerLab window. SS-EMG was continuously recorded during the cystometry.

Drug Administration

An intravenous cannula was connected to the ear vein for vehicle (saline) or drug administration. The drugs were freshly prepared before each experiment in saline. Drugs were administered intravenously in a volume of 1 ml. followed by 1 ml. flush of physiological saline.

Statistical Analysis

All values were expressed as the mean±SEM. Duloxetine or HP749 effects were analyzed and compared with control (saline) values using Wilcoxon rank test. Mann-Whitney U test was performed to compare the effects of duloxetine and HP-749. Statistical significance was considered at p<0.05.

Results

Reproducible cystometric patterns were obtained. Intravenous injection of the vehicle produced no effects on either cystometric parameters or SS-EMG activity.

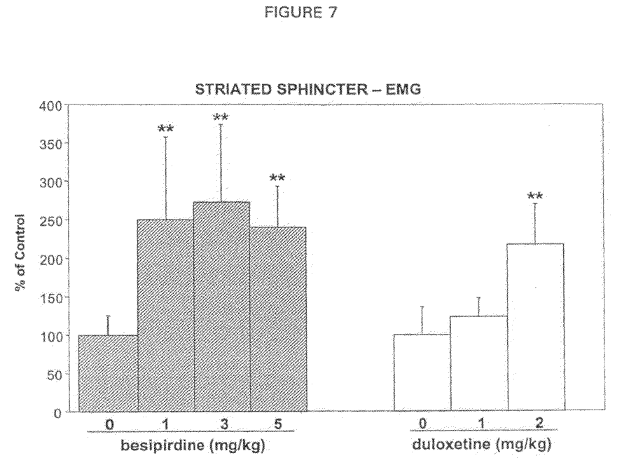

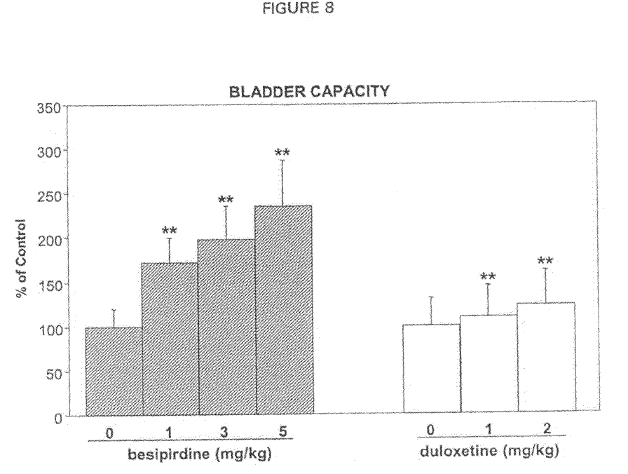

Effects of Duloxetine, as shown in FIGS. 7 and 8

Duloxetine increased dose-dependently the bladder capacity (p<0.01). Moreover, SS-EMG activity was significantly increased by duloxetine at 2 mg./kg (p<0.01).

Effects of HP749, as shown in FIGS. 7 and 8

HP749 dose-dependently increased the bladder capacity, from 172% of the control at 1 mg/kg to 235% at 5 mg/kg (p<0.01). SS-EMG was also increased 2.5-fold at all doses tested (p<0.01).

The effects of HP749 on bladder capacity and SS-EMG were significantly higher than those of duloxetine (p<0.05).

In conclusion, these results show that HP749 enhances the activity of the striated sphincter from 1 mg/kg and, in parallel, increases the bladder capacity. This indicates that HP749 enhances urethral closure. As compared to duloxetine, HP749 had a higher effect on both SS-EMG activity and cystometric parameters.

1. A method of treatment of stress urinary incontinence and of mixed urinary incontinence, comprising:
administering a medicinal product corresponding to formula (I): 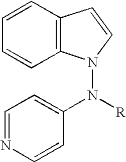
in which R represents hydrogen or a group chosen from alkyl, alkylene, alkylidyne, cycloalkyl, cycloalkylene, cycloalkylidyne and —CONH₂ groups, and —COR′ and —COOR′ groups, where R′ is chosen from alkyl, alkylene, alkylidyne, cycloalkyl, cycloalkylene and cycloalkylidyne groups, it being possible for said groups R and/or R′ to be substituted and/or interrupted with —O—, —COO—, —OCO—, —NHCO— or —CONH— functions, or a pharmaceutically acceptable salt of said compound,
to a subject that has symptoms of stress urinary incontinence or mixed urinary incontinence.

2. The method according to claim 1, wherein R is the n-propyl group.

3. The method according to claim 1, wherein two compounds corresponding to formula (I) are combined.

4. The method according to claim 3, wherein a compound of formula (I) in which R represents H and a compound of formula (I) in which R represents the n-propyl group are combined.

5. The method according to claim 2, wherein two compounds corresponding to formula (I) are combined.
